(19) United States
(12) Patent Application Publication
(10) Pub. No.: US 2011/0034849 A1
(43) Pub. Date: Feb. 10, 2011

(54) Contoured eye patch

(76) Inventor: Andrell Cooks, Columbus, OH (US)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/848,813

(22) Filed: Aug. 2, 2010

Related U.S. Application Data
(60) Provisional application No. 61/231,707, filed on Aug. 6, 2009.

(51) Int. Cl. A61F 13/12

(52) U.S. Cl. 602/74

(57) ABSTRACT

A disposable self-adhering covering for an eye of a human comprising an opaque, thin, flexible, air permeable fabric layer having an upper surface and a lower surface bonded to top side of a less flexible generally ovoid-shaped generally flat ring defining a central opening. The ring comprising a bottom side comprising an adhesive that allows the covering to be removably attachable to the skin of a human and an outer contoured edge that causes the ring to flex away from the skin such that the fabric layer is held away from the eye when applied to the human. The covering extends over the eye with the ring attached to the skin from a point at the medial orbital margin of the eye to a point at the lateral orbital margin of the eye with the central opening over the eye socket.

### CROSS-REFERENCES TO RELATED APPLICATIONS

The present application is a continuation of and claims priority to provisional patent application No. 61/231,707 filed on Aug. 6, 2009, which is hereby incorporated by reference in its entirety.

### FIELD OF THE INVENTION

The present invention relates generally to eye patches and more particularly to a self adhering opaque eye patch that allows air flow to the eye socket.

### BACKGROUND OF THE INVENTION

Of the many challenges facing people who have lost an eye is finding an eye covering that is inexpensive, comfortable and attractive. Existing eye patches may be maintained in place by a strap or by an adhesive that holds the patch to the skin. Prior art eye patches using adhesive typically have had problems remaining attached to the skin around the eye area for extended lengths of time. Existing eye patches also have problems fitting to the face at the medial orbital margin where the eye socket meets the nose. If the patch is bulky or protrudes too far from the face, the other eye may have portions of vision obstructed by the patch. Most existing eye patches make contact the eye lid to hold the eye shut. As a result, these patches are not comfortable. In addition, most existing eye patches do not provide adequate air circulation to the eye socket. Without air flow, the patch causes heat to be retained and moisture to build up under the eye patch, which can lead to irritation and potential medical problems.

U.S. Pat. No. 4,682,371 to Heitman discloses a socket-shaped, lightweight, substantially rigidly framed eye patch that is adhesively secured over an eye. The adhesion is by mounting tabs that only secure the eye patch to the face at given points, causing gaps between the patch and the skin such that the eye socket may be visible.

U.S. Pat. No. 4,951,658 to Morgan, et al., discloses an eye patch having a pressure sensitive adhesive material for attaching to the skin surrounding the eye. The patch includes an inner member that contacts the eyelid. Morgan does not provide any way for air to circulate under the patch to relieve sweaty or humid conditions and provide air flow.

U.S. Pat. No. 5,661,850 to Martinique discloses an eye patch having an upper edge spaced apart from the forehead of the user to provide air circulation for the eye. Such a gap between the patch and the skin does not provide complete concealment of the socket.

U.S. Pat. No. 4,944,040 to Riedel, et al., discloses a self-adherent moisture vapor permeable eye patch that contacts the eye lid. Riedel uses high bond tapes that may tear the delicate skin around the eye upon removal.

U.S. Pat. No. 4,979,811 to Boyer discloses a patch that only covers the eyelid having an and outer layer that may be provided with a decorative design.

An inexpensive, comfortable eye patch is needed that stays securely attached by an gentle adhesive to the skin for at least a full day that is safe to apply to skin, does not cause skin problems, and will not peel the skin away upon removal. An eye patch is needed that follows the contour of the shape of the nose and eye socket, that does not come in contact with the eye lid or lashes, and allows the remaining eye an unobstructed field of vision. An eye patch is needed that allows sufficient air flow to the eye socket while concealing the socket.

### BRIEF DESCRIPTION OF THE INVENTION

The present invention is a disposable self-adhering covering for an eye of a human. The covering comprises an opaque, thin, flexible, air permeable fabric layer having an upper surface and a lower surface and a less flexible generally ovoid-shaped generally flat ring defining a central opening. The ring has a top side and a bottom side. The top side of the ring is sealed to an outer portion of the lower surface of the fabric layer. The bottom side of the ring has a gentle but strong adhesive that allows the covering to be removably attachable to the skin around the eye socket of a human. The bottom side of the ring has an outer contoured edge and an inner edge. When applied to a human, the covering extends over the eye with the ring attached to the skin from a point at the medial orbital margin of the eye to a point at the lateral orbital margin of the eye with the central opening over the eye socket. The contoured edge of the ring causes the ring to flex away from the skin from the outer to the inner edge such that the fabric layer is held away from the eye.

In an embodiment, the ring is comprised of felt. In an embodiment, the upper surface of the fabric layer includes a decoration. In an embodiment, the dimensions of the covering are about 8 cm by about 5.5 cm and the distance from the opening to the edge of the ring is 1 cm.

As used herein, “approximately” means within plus or minus 25% of the term it qualifies. The term “about” means between ½and 2 times the term it qualifies.

The compositions and methods of the present invention can comprise, consist of, or consist essentially of the essential elements and limitations of the invention described herein, as well as any additional or optional ingredients, components, or limitations described herein or otherwise useful in compositions and methods of the general type as described herein.

All references to singular characteristics or limitations of the present invention shall include the corresponding plural characteristic or limitation, and vice versa, unless otherwise specified or clearly implied to the contrary by the context in which the reference is made.

### BRIEF DESCRIPTION OF THE DRAWINGS

FIG. 1 is a top view of the invention.

FIG. 2 is a bottom view of the invention.

FIG. 3 is an exploded top perspective view of the invention.

FIG. 4 is a bottom view of the invention.

FIG. 5 is a perspective view of an alternate embodiment of the invention with a strap.

FIG. 6 is a diagrammatic view showing placement of the covering on the eye.

FIG. 7 is a perspective view showing an alternate embodiment having a design and in place on a user.

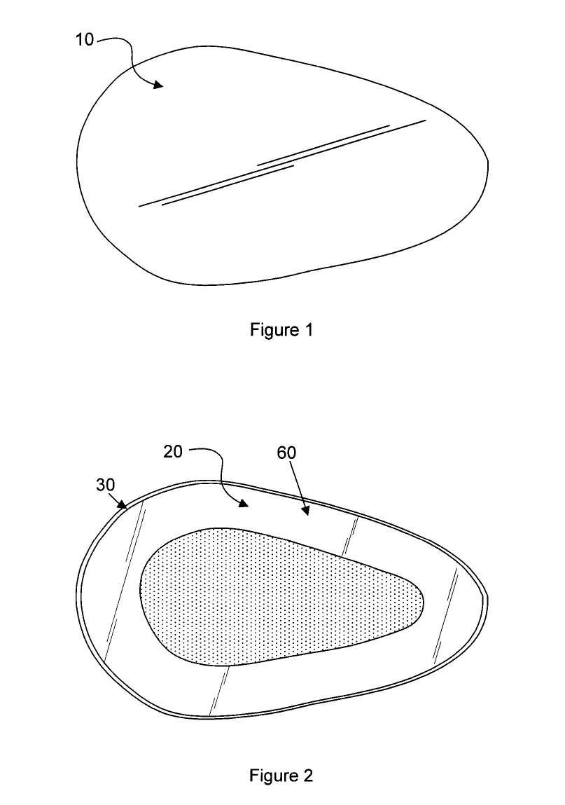

While the views of the Figures depict a right side embodiment, one skilled in the art would readily understand that the invention includes an embodiment for the left side that is a mirror image of the embodiment depicted.

### DETAILED DESCRIPTION OF THE INVENTION AND BEST MODE

The present invention has a pliable top layer 10 connected to a ring 20. The pliable layer 10 can be made from any opaque lightweight breathable material that can be formed in sheets, such as textiles and the like. In an embodiment, the pliable layer 10 is made from a thin layer of a textile. In an embodiment, the textile is a cotton, pima cotton, seersucker, linen, wool, merino, cashmere, breathable polyester and blends thereof As shown in FIG. 7, the viewable surface of the pliable layer may include a design. Any design or decoration that fits on the surface and does not impede airflow may be used. The pliable top layer 10 is attached to a ring 20. In an embodiment, the ring is formed from a thin felt, although any similar material that is relatively stiff yet elastic, soft to the touch, strong, thin and porous may be used to form the ring. The ring comprises an outer area surrounding a central cut out area. The center of the ring is maximized to provide the greatest amount of airflow to the eye socket. In an embodiment, the ring 20 is stamped or cut from acid-free iron-on felt available at most sewing suppliers. The iron-on adhesive is heat activated to bond the ring to the pliable top layer 10.

As shown in FIG. 2, the ring 20 comprises a beveled edge 30 located at an outer edge of the side of the ring that contacts the face. The shape of the ring and the shape of the pliable layer generally follow the contour of the bony structure of the eye socket (right and left).

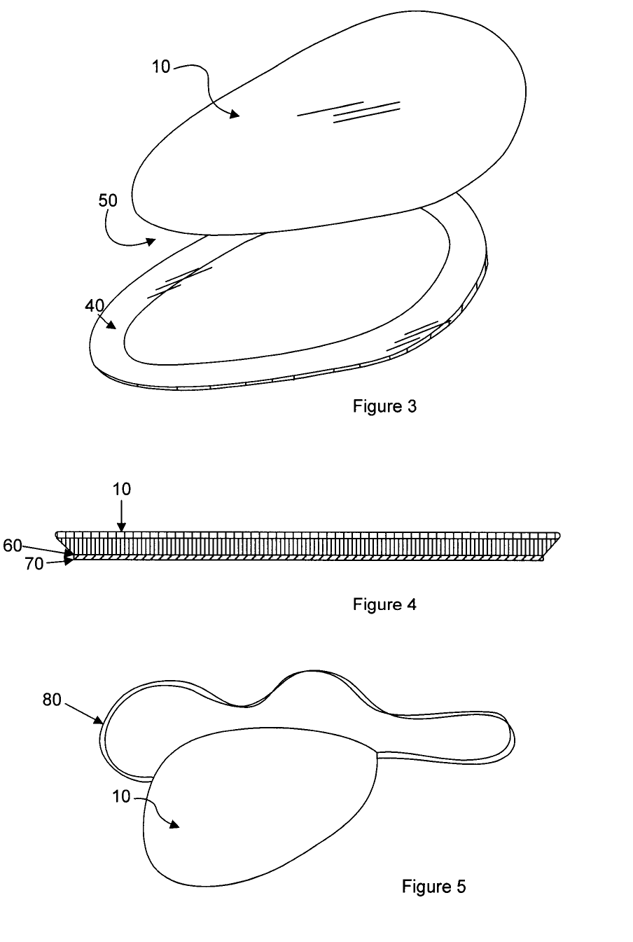

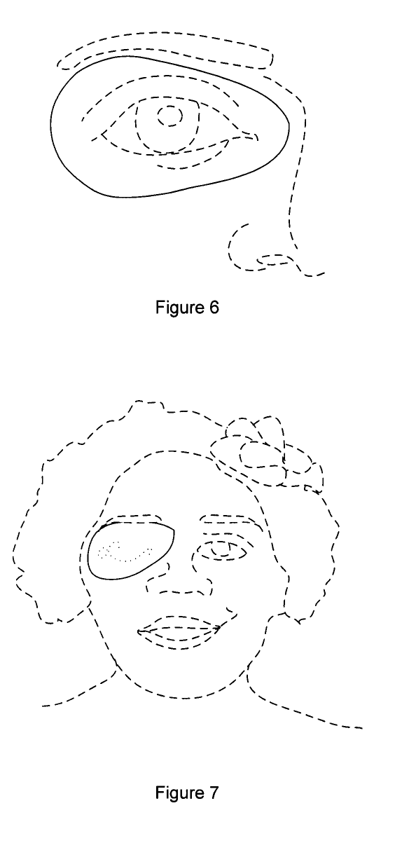

As best seen in FIG. 3, the top side 40 of the ring is bonded to an outer edge of the bottom side 50 of the pliable layer 10. Referring to FIG. 4 (where the thickness of the elements are only provided for clarity of illustration) the bottom side of the ring 60 comprises an adhesive layer 70 bonded to the surface of the ring to form a contact surface that holds the eye patch in place over an eye without straps or bands. The adhesive layer 70 may be applied to the bottom side 60 as a layer or in selected areas or as a grid. The adhesive layer is made from any substance that will attach to the skin, is peeled off easily and provides strong bonding power while being lightweight and not adding stiffness.

Suitable adhesives include spray adhesives, fabric glues, adhesive tapes, and the like. In an embodiment, the adhesive is a releasably attachable pressure sensitive adhesive backing having a liner with a release coating on one side for covering the adhesive coating. The liner is formed from a disposable paper or plastic liner that is removed immediately prior to adhering the patch to the skin. The adhesive is preferably assembled from commercially available products. For example, transparent polypropylene film having a silicone release coating on one face is commercially available from the Mobil Chemical Corporation. A paper liner having a release coating and an acrylic adhesive laminated to one side is commercially available from the 3M Corporation. Alternatively, the present invention may comprise a band 80 extending from opposite outer edges of the ring to circle the head to provide additional secure placement of the patch rather than the adhesive.

The present invention has the following advantages:

It is formed in a sheet and is thin, so has a small volume.

It is made from a textile, so it breathes and a user will not feel damp.

It is made inexpensively and can be discarded after use.

The covering, when applied to the eye, is able to adhere for approximately 48-72 hours (about three days). The covering may be worn during the daytime and through the night. Alternatively, the banded embodiment may be used overnight. Use of the banded version provides additional circulation to the socket.

While the adhesive of the covering will fail if saturated with water, the adhesive remains attached during work-outs where sweat is generated.

The foregoing description of the invention has been presented for purposes of illustration and description. It is not intended to be exhaustive nor to limit the invention to the precise form disclosed. Many alternatives, modifications, and variations will be apparent to those skilled in the art. For instance, the present invention could be made from other materials and shaped to cover a smaller or larger area of the face. It is to be understood that the terms used herein are merely descriptive, rather than limiting, and that various changes may be made without departing from the spirit of the scope of the invention.

What is claimed is:

1. A disposable self-adhering covering for an eye of a human comprising:
(a) an opaque, thin, flexible, air permeable fabric layer having an upper surface and a lower surface; and
(b) a less flexible generally ovoid-shaped generally flat ring defining a central opening; said ring comprising a top side and a bottom side, said top side of the ring sealed to an outer portion of the lower surface of the fabric layer,
said bottom side of the ring comprising (i) an adhesive, said adhesive allowing the covering to be removably attachable to the skin of the human, (ii) an outer contoured edge, and (iii) an inner edge, said contoured edge of the ring causing the ring to flex away from the skin from the outer to the inner edge such that the fabric layer is held away from the eye when applied to the human and the covering extends over the eye with the ring attached to the skin from a point at the medial orbital margin of the eye to a point at the lateral orbital margin of the eye with the central opening over the eye socket.

2. The covering of claim 1 wherein the ring is comprised of felt.

3. The covering of claim 1 wherein the upper surface of the fabric layer includes a decoration.

4. The covering of claim 1 wherein the dimensions of the covering are about 8 cm by about 5.5 cm and the distance from the opening to the edge of the base is 1 cm.
